(19) United States
(12) Patent Application Publication
(10) Pub. No.: US 2025/0073211 A1
(43) Pub. Date: Mar. 6, 2025

(54) USE OF ERGOTHIONEINE TO SUPPRESS DELETERIOUS EFFECTS OF SENESCENCE

(71) Applicant: NANJING NUTRABUILDING BIO-TECH CO., LTD., Nanjing (CN)

(72) Inventors: Joseph L. EVANS, Nanjing (CN); Qiru FAN, Nanjing (CN); Shawn WELLS, Nanjing (CN); Kylin LIAO, Nanjing (CN)

(21) Appl. No.: 18/948,449

(22) Filed: Nov. 14, 2024

Related U.S. Application Data
(63) Continuation of application No. PCT/CN2022/093791, filed on May 19, 2022.

(51) Int. Cl. A61K 31/4172

(52) U.S. Cl. CPC A61K 31/4172

(57) ABSTRACT

The present invention relates to a method for suppressing deleterious effects of senescence in a mammal. The method includes administration to the mammal of a composition comprising therapeutically effective amount of ergothioneine, or a pharmaceutically acceptable salt, acid, ester, analog or derivative thereof. In some embodiments, the composition suppressing deleterious effects of senescence is by down-regulation of p21 and/or SAβG.

### CROSS REFERENCE TO RELATED APPLICATION

This application is a continuation application of International Patent Application No. PCT/CN2022/093791, filed on May 19, 2022, the contents of which are incorporated herein by reference in its entirety.

### BACKGROUND OF THE INVENTION

Senescence is the gradual deterioration of functional characteristics in living organisms. It is characterized by the declining ability to respond to stress, increased homeostatic imbalance, and increased risk of aging-associated diseases including cancer and heart disease. Senescence is the inevitable fate of almost all multicellular organisms with germ-soma separation, but it can be delayed.

Ergothioneine (“Ergo”) is a naturally occurring amino acid and is a thiourea derivative of histidine, containing a sulfur atom on the imidazole ring. It occurs in relatively few organisms, notably Actinobacteria, Cyanobacteria, and certain fungi. In humans, ergothioneine is acquired exclusively through the diet and accumulates in erythrocytes, bone marrow, liver, kidney, seminal fluid, and eyes.

Although the effect of ergothioneine in vivo is an active area of research, its physiological role in humans is undetermined.

### SUMMARY OF THE INVENTION

In a first aspect, the present invention provides a method for suppressing deleterious effects of senescence in a mammal. The method includes administration to the mammal of a composition comprising therapeutically effective amount of ergothioneine, or a pharmaceutically acceptable salt, acid, ester, analog or derivative thereof.

In another aspect, the present invention provides a method of down-regulation of p21 and/or SaβG in a mammal in need thereof, comprising administration to the mammal of a composition comprising a therapeutically effective amount of ergothioneine or a pharmaceutically acceptable salt, acid, ester, analog or derivative thereof. In some embodiments, the method is senostatic or suppresses senescence.

In some embodiments, the senescence phenotype is reflected through HDFn. In some embodiments, the senescence phenotype is reflected through HDFn cell lines.

In some embodiments, the mammal is a human, horse, cattle or other ruminants, pig, or a pet.

In some embodiments, the composition is in a form of food, drink, nutritional composition, or pharmaceutical composition.

In some embodiments, the composition is in a form of solution, liquid suspension, parenteral solution, injectable solution, tablet, pill, granule, powder, film, (micro) capsule, aerosol, tonic, syrup, beverage, or dietary supplement.

In some embodiments, the administration is at least once a day or more times a day.

In some embodiments, the administration is via oral, intravenous injectable, intramuscular injectable, intraperitoneal, intranasal, rectal, or sublingual route.

In some embodiments, the administration of the composition is with a daily dose of ergothioneine in the range of 2-2,000 mg, 2-500 mg, 2-200 mg, 2-150 mg, 5-100 mg, or 5-50 mg.

In some embodiments, the daily dose is administered in divided doses or a single dose. In some embodiments, the administration is for a week or more, or for months or year.

In some embodiments, the composition suppressing deleterious effects of senescence is by down-regulation of p21 and/or SAβG.

In a second aspect, the present invention provides a composition comprising therapeutically effective amount of ergothioneine, or a pharmaceutically acceptable salt, acid, ester, analog or derivative thereof, for suppressing deleterious effects of senescence in a mammal in need thereof.

In some embodiments, the mammal is a human, horse, cattle or other ruminants, pig, or a pet.

In some embodiments, the composition is in a form of food, drink, nutritional composition, or pharmaceutical composition.

In some embodiments, the composition is in a form of solution, liquid suspension, parenteral solution, injectable solution, tablet, pill, granule, powder, film, (micro) capsule, aerosol, tonic, syrup, beverage, or dietary supplement.

In a third aspect, the present invention provides a method for preparing any one of the compositions disclosed herein.

In a fourth aspect, the present invention provides use of any one of the compositions disclosed herein for suppressing deleterious effects of senescence in a mammal.

In some embodiments, ergothioneine is administrated at a daily dose of 2-2000 mg, 2-500 mg, 2-200 mg, 2-150 mg, 5-100 mg, or 5-50 mg.

In some embodiments, the administration is at least once a day or more times a day.

In some embodiments, the administration is by oral, intravenous injectable, intramuscular injectable, intraperitoneal, intranasal, rectal, or sublingual route.

In some embodiments, the composition suppressing deleterious effects of senescence is through down-regulation of p21 and/or SAβG.

These and other features, aspects, and advantages of the present invention will become better understood with reference to the following description and appended claims.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C are bar charts showing that the percentage of HDFn cells, which were treated at passages 22, 24, and 26 with EGT at various concentrations (0, 0.5, 3.0and 30 μM) and H₂O₂, respectively, are positive for Ki67, p21, or p16 proteins. FIG. 1D shows the percentage of β-galactosidase positive HDFn cells with the same treatments. p22 refers to passage 22, p24 refers to passage 24, and p26 refers to passage 26. For each graph of FIGS. 1A-1D, a two-way ANOVA was performed with Bonferroni post-hoc analysis against the 0 μM treatment. * p<0.05, ** p<0.01, *** p<0.001. FIG. 1E shows representative images of p21 immunostaining of HDFn cells treated at passage 22 with EGT at various concentrations (0, 0.5, 3.0 and 30 μM). FIG. 1F shows representative images of SAβG staining of HDFn cells treated at passage 26 with EGT at various concentrations (0, 0.5, 3.0 and 30 μM). Images were taken at 100× magnification.

FIG. 2A are bar charts showing cell counts of HDFn cells treated at passage 31 with EGT at various concentrations (0, 3, 30, and 300 μM), DMSO, Fisetin (FIS), and Navitoclax (NAV). Positive controls for senolytics function are 600 μM Fisetin and 13 μM Navitoclax. DMSO is the vehicle control for Fisetin. d0 refers to day 0 of seeding, d0-3 refers to 3 days after treatment on cells seeded on day 0, d6-9 refers to 3 days after treatment on cells seeded on day 6, and d9-12 refers to 3 days after treatment on cells seeded on day 9. FIG. 2B shows representative images for cell count with various treatments. Images were taken at 100× magnification.

FIGS. 3A-3C are bar charts showing that the percentage of HDFn cells, which were treated at passage 31 with EGT at various concentrations (0, 0.5, 3.0 and 30 μM), DMSO, Fisetin (FIS), and Navitoclax (NAV), respectively, are positive for Ki67, p21, or p16 proteins. FIG. 3D shows the percentage of β-galactosidase positive HDFn cells with the same treatments. d0-3 refers to 3 days after treatment on cells seeded on day 0, d6-9 refers to 3 days after treatment on cells seeded on day 6, and d9-12 refers to 3 days after treatment on cells seeded on day 9. For each graph of FIGS. 3A-3D, a two-way ANOVA was performed with Bonferroni post-hoc analysis against the 0 μM treatment. * p<0.05, *** p<0.001.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

In a first aspect, the present invention provides a method for suppressing deleterious effects of senescence in a mammal. The method includes administration to the mammal of a composition comprising therapeutically effective amount of ergothioneine, or a pharmaceutically acceptable salt, acid, ester, analog or derivative thereof.

In a second aspect, the present invention provides a composition comprising therapeutically effective amount of ergothioneine, or a pharmaceutically acceptable salt, acid, ester, analog or derivative thereof, for suppressing deleterious effects of senescence in a mammal in need thereof.

In a third aspect, the present invention provides a method for preparing any one of the compositions disclosed herein.

In a fourth aspect, the present invention provides use of any one of the compositions disclosed herein for suppressing deleterious effects of senescence in a mammal.

At macro lever, senescence is the gradual deterioration of functional characteristics in living organisms. It is characterized by the declining ability to respond to stress, increased homeostatic imbalance, and increased risk of aging-associated diseases including cancer and heart disease.

At micro level, senescence refers to the phenomenon whereby normal nonmalignant cells stop dividing in vitro. Senescence at micro level is also call replicative senescence. Replicative senescence is induced by telomere shortening. With each round of DNA replication, telomeres are progressively shortened, eventually reaching a critical length which prevents further replication, thereby halting cell division. Critically short, uncapped telomeres initiate a DNA damage response which triggers senescence.

Certain proteins have been used as indicators of senescence induction or progression. Ki67 is a standard nuclear marker for cell proliferation. Proliferating cells results in stronger Ki67 staining, while senescent and quiescent cells do not show Ki67. P21 and p16 are early and late cell cycle checkpoint markers, respectively. Upon senescence induction by various inducers, p21 is first recruited to the nucleus to prevent further proliferation. If the senescence-inducing stressor is not alleviated, the checkpoint is then reinforced by p16 recruitment. β-galactosidase is a lysosomal enzyme that is upregulated during cessation of cell proliferation, including both senescence and contact-inhibition. The individual presence of each marker is insufficient to proclaim a senescence phenotype. The progression of Ki67, p21, p16, and SAβG through senescence development in the MRC5 and IMR90 human fibroblast cells lines have been alluded to in many publications, but never explicitly established and shown (e.g., Correia-Melo et al., 2016, Mitochondria are required for pro-ageing features of the senescent phenotype. EMBO J, 35 (7), 724-742). The consensus profile for these markers during senescence induction is an immediate downregulation of Ki67, followed by nuclear localization of p21 between one and three days after induction, followed by p16 recruitment and increased SAβG activity between seven to ten days after induction.

Ergothioneine is a naturally occurring amino acid and is a thiourea derivative of histidine, containing a sulfur atom on the imidazole ring. This compound occurs in relatively few organisms, notably Actinobacteria, Cyanobacteria, and certain fungi. In humans, ergothioneine is acquired exclusively through the diet and accumulates in erythrocytes, bone marrow, liver, kidney, seminal fluid, and eyes. In vitro, ergothioneine requires a specific transporter, ETT, also known as OCTN1 (gene symbol SLC22A4), to enter cells. Although the effect of ergothioneine in vivo is an active area of research, its physiological role in humans is undetermined (Cheah et al., “Ergothioneine; antioxidant potential, physiological function and role in disease,” Biochimica et Biophysica Acta (BBA)-Molecular Basis of Disease. 2012, 1822 (5):784-93).

As used herein, a “therapeutically effective amount” refers to a sufficient amount of ergothioneine for suppressing deleterious effects of senescence in a mammal, at a reasonable benefit/risk ratio applicable to any medical treatment. It will be understood, however, that the total daily usage of ergothioneine may be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of ergothioneine employed; the duration of the treatment; drugs used in combination or coincidental with ergothioneine; and like factors well known in the medical arts. For example, it is well known within the skill of the art to start doses of the compound at levels lower than those required to achieve the desired therapeutic effect and to gradually increase the dosage until the desired effect is achieved. In addition, a “therapeutically effective amount” is the amount that will elicit the biological or medical response of a tissue, system, or subject that is being sought by a researcher or clinician, and in particular elicit some desired therapeutic or prophylactic effect for suppressing deleterious effects of senescence in a mammal.

One of skill in the art recognizes that an amount may be considered therapeutically “effective” even if the condition is not totally eradicated or prevented, but it or its symptoms and/or effects are improved or alleviated partially in the subject. Various indicators for determining the effectiveness of a method for suppressing deleterious effects of senescence in a mammal are known to those skilled in the art.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

In some embodiments, the composition comprises from about 5% to about 10%, 20%, 30%, 40%, 50%, 60%, 70%, 80%, 90%, or 95% by weight of ergothioneine, and preferably from about 30% to about 90% by weight of ergothioneine, based upon the total weight of the composition taken as 100% by weight.

Other ingredients may be included in the claimed composition, such as other active agents, preservatives, buffering agents, salts, a pharmaceutically acceptable carrier, or other pharmaceutically acceptable ingredients.

As used herein, a “carrier” refers to a compound that facilitates the incorporation of a compound into cells or tissues. For example, without limitation, dimethyl sulfoxide (DMSO), Ethanol (EtOH), or PEG400 is a commonly utilized carrier that facilitates the uptake of many organic compounds into cells or tissues of a subject.

“Mammal” includes, without limitation, mice, rats, rabbits, guinea pigs, dogs, cats, sheep, goats, cattle, horses, primates, such as monkeys, chimpanzees, and apes, and, in particular, humans.

In some embodiments, the mammal is a human, horse, cattle or other ruminants, pig, or a pet. “Pet” includes, without limitation, mice, rats, rabbits, guinea pigs, dogs, cats, fish, reptiles, and sheep.

In some embodiments, the composition is prepared in a form of food, drink, nutritional composition, or pharmaceutical composition.

The term “pharmaceutical composition” refers to a mixture of ergothioneine with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration. A pharmaceutical composition is suitable for human and/or veterinary applications.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

The pharmaceutical compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include ergothioneine formulated in a compatible pharmaceutical carrier may also be prepared, placed in an appropriate container, and labeled for treatment of an indicated condition.

In some embodiments, the composition is in a form of solution, liquid suspension, parenteral solution, injectable solution, tablet, pill, granule, powder, film, (micro) capsule, aerosol, tonic, syrup, beverage, or dietary supplement.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant, for example, sugar cane, corn, and maple.

In some embodiments, the administration is at least once a day or more times a day.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. One may also administer the composition in a local rather than systemic manner, for example, via injection of the compound directly into the infected area, often in a depot or sustained release formulation. Furthermore, one may administer the composition in a targeted drug delivery system, for example, in a liposome coated with a tissue specific antibody. The liposomes will be targeted to and taken up selectively by the organ.

In some embodiments, the administration is through various routes selected from oral, intravenous injectable, intramuscular injectable, intraperitoneal, intranasal, rectal, or sublingual route.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

In some embodiments, the administration of the composition is by oral with a daily dose of ergothioneine in the range of 2-2000 mg. In some embodiments, the administration of the composition is by oral with a daily dose of ergothioneine in the range of 5-500 mg. In some embodiments, the administration of the composition is by oral with a daily dose of ergothioneine in the range of 5-25 mg.

The dosage may range broadly, depending upon the desired effects and the therapeutic indication. The daily dosage regimen for an adult human patient may be, for example, an oral dose of between 0.01 mg and 3000 mg of ergothioneine, preferably between 1 mg and 700 mg, e.g. 5 to 200 mg, or between about 0.1 mg and about 1,000 mg of ergothioneine per kg of body weight of the subject. The dosage may be a single one or a series of two or more given in the course of one or more days, as is needed by the subject. In some embodiments, the compounds are administered for a period of continuous therapy, for example for a week or more, or for months or years. In some embodiments, ergothioneine, or a pharmaceutically acceptable salt thereof, can be administered less frequently compared to the frequency of administration of an agent within the standard of care. In some embodiments, ergothioneine, or a pharmaceutically acceptable salt thereof, can be administered one time per day. In some embodiments, the total time of the treatment regime with ergothioneine, or a pharmaceutically acceptable salt thereof, can be less compared to the total time of the treatment regime with the standard of care.

In instances where human dosages for ergothioneine have been established for at least some condition, those same dosages may be used, or dosages that are between about 0.1% and 500%, more preferably between about 25% and 250% of the established human dosage. Where no human dosage is established, as will be the case for newly discovered pharmaceutical compositions, a suitable human dosage can be inferred from ED50 or ID50 values, or other appropriate values derived from in vitro or in vivo studies, as qualified by toxicity studies and efficacy studies in animals.

As used herein, the term “ED50” refers to the dose that produces the desired effect in 50% of the population, or median effective dose.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety that are sufficient to maintain the modulating effects, or minimal effective concentration (MEC). Dosages necessary to achieve the MEC will depend on individual characteristics and route of administration. However, HPLC assays or bioassays can be used to determine plasma concentrations. Dosage intervals can also be determined using MEC value. Compositions should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In some embodiments, the daily dose is administered in divided doses or a single dose.

In some embodiments, the composition suppressing deleterious effects of senescence is by down-regulation of p21 and/or SAβG.

Upon senescence induction by various inducers, p21 is first recruited to the nucleus to prevent further proliferation. β-galactosidase is a lysosomal enzyme that is upregulated during cessation of cell proliferation. Thus, down-regulation of p21 and/or SAβG could suppress the deleterious effects of senescence.

### DEFINITIONS

Unless defined otherwise, all technical and scientific terms used herein have the same meaning as is commonly understood by one of ordinary skill in the art. In the event that there are a plurality of definitions for a term herein, those in this section prevail unless stated otherwise.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “individual,” “patient,” or “subject” are used interchangeably. None of the terms require or are limited to situation characterized by the supervision (e.g., constant or intermittent) of a health care worker (e.g. a doctor, a registered nurse, a nurse practitioner, a physician's assistant, an orderly, or a hospice worker).

“Replicative senescence” refers to the phenomenon whereby normal nonmalignant cells stop dividing in vitro.

“Senostatics” refers to selectively suppressing the deleterious effects of senescence. “Senostatic profile” refers to the expression pattern of certain protein markers in the process of senostatics.

“Senolytics” refers to selectively eliminating senescent cells. “Senolytic profile” refers to the expression pattern of certain protein markers in the process of senolytics.

Control: A control is an individual or a group of samples used as a standard of comparison for checking the results of a survey or experiment. In some context, a control is expressed as a reference.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. The term “therapeutically effective” should be understood to have a definition corresponding to ‘having a therapeutic effect.

Any titles or subheadings used herein are for organization purposes and should not be used to limit the scope of embodiments disclosed herein.

All publications and patent applications cited in this specification are herein incorporated by reference as if each individual publication or patent application were specifically and individually indicated to be incorporated by reference.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

MitoPrime (L-Ergothioneine (EGT); lot number MP20200906) was received from Nanjing Nutrabuilding Bio-tech, Nanjing, China (NNB) as a 1 g powder. MitoPrime (lot number MP20200906) was produced using proprietary fermentation technology by NNB, and added to the culture medium to achieve the indicated final concentrations.

The powder was decanted into a plastic bijou and stored at room temperature away from sunlight. Stock solutions of 0.5 mM, 3.0 mM, 30 mM, and 300 mM in DMEM were made fresh every week and filter sterilized. The stock solutions were kept at 4° C.

### HDFn (Neonatal Human Dermal Fibroblast) Cells

HDFn cells (C0045C, Thermo Scientific, US) were obtained and proliferated to passage 32 (population doubling 58) in standard DMEM proliferation medium (10% FBS) at standard culture conditions (37°° C., 20% O₂, 5% CO₂). At each passage, cells were cryopreserved and banked. The cells were deemed replicatively senescent at passage 32. When required, cryovial contents was thawed gradually into 5 ml pre-warmed 10% FBS media with gentle pipetting and centrifuged. The supernatant was discarded, and the cell pellet was resuspended into 10% media and cells were counted using a hemocytometer and trypan blue.

### Senostatic Profile Assay

Treatment conditions tested in this assay were 0, 0.5, 3.0 and 30 μM of EGT as a constant supplementation in standard DMEM (10% FBS). HDFn cells were thawed at passage 20 and seeded at 1.5×10⁵ cells per flask. Three flasks per condition were seeded. Media was changed every three days. At passages 22, 24, and 26, the HDFn cells were seeded onto 48-well plates at 10,000 cells per well in triplicates per flask, and cultured in DMEM media containing 0, 0.5, 3.0, and 30 μM of EGT, respectively. The cells were proliferated until 70% confluence or for 7 days. The cells were then washed with PBS and fixed with 2% PFA, and stored at 4° C. until immunostaining. For β-galactosidase staining, the staining solution was added onto the cells immediately after fixation and incubated at 37° C. for 24 h. The staining solution was removed, and the wells filled with PBS until imaging. As a positive control for senescence phenotype, one set of wells from the untreated flasks were seeded. At 70% confluence, the cells were treated with 600 μM of hydrogen peroxide in serum-free DMEM for 2 h, then incubated with standard DMEM proliferation medium for 5 days to allow the senescence phenotype to develop. The cells were fixed and stained in the same manner as the other treatment conditions.

Cell pellets for each flask were collected for RNA analysis and stored at −80° C. until further action.

### Senolytic Profile Assay

Treatment conditions tested in this assay were 0, 3, 30 and 300 μM of EGT as an acute supplementation over 3 days in standard DMEM (10% FBS). Positive controls for senolytic activity were 13 μM of Navitoclax and 600 μM of Fisetin. A 0.1% DMSO condition was introduced as a vehicle control for Fisetin. At passage 31, the HDFn cells were seeded at 10,000 cells per well in triplicate wells onto 48-well plates. After 2 days of acclimatization, cells were treated for 3 days with the conditions above. The cells were then washed with PBS and fixed with 2% PFA. The wells were filled with PBS and stored at 4° C. until immunostaining. For β-galactosidase staining, the staining solution was added onto the cells immediately after fixation and incubated at 37° C. for 24 h. The staining solution was removed, and the wells filled with PBS until imaging. This was repeated for timepoints day 0 to 3, day 6 to 9, and day 9 to 12.

### Histological Procedures

The panel of senescence markers, Ki67, p16 and p21 were analyzed by immunohistochemical staining using an HRP/DAB kit (ab64264, Abcam, UK).
Antibody details
   1) Ki67—1:1000 dilution, anti-rabbit, ab92742, Abcam;
   2) p16—1:500 dilution, anti-rabbit, ab 108349, Abcam;
   3) p21—1:100 dilution, anti-rabbit, ab 109542, Abcam.

The cells were counterstained with hematoxylin.

The senescence-associated β-galactosidase solutions and staining was performed according to Dimri et al, A biomarker that identifies senescent human cells in culture and in aging skin in vivo, Proc. Natl. Acad. Sci. 1995 Sep. 26; 92 (20): 9363-9367. The cells were counterstained with Nuclear Fast Red.

All staining were imaged on an EVOS XL core microscope at 100× magnification.

### Quantification, Analysis, Statistics

The images were white balanced using a macro on ImageJ. Cells were manually counted using the cell counter plugin and recorded. Graphs and statistics were created using GraphPad Prism 5. For each dataset, a two-way ANOVA was performed with Bonferroni posthoc test against the untreated control. P-values on the graphs are shown as *p<0.05, ** p<0.01, *** p<0.001.

### RESULTS

### Senostatic Profile Assay

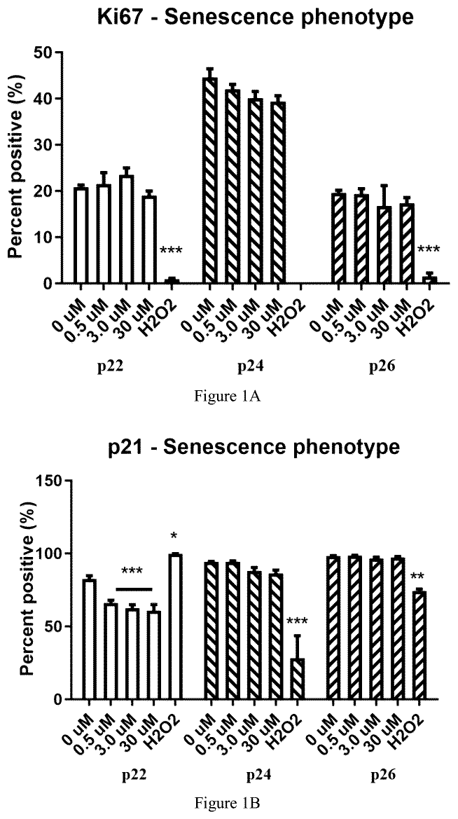

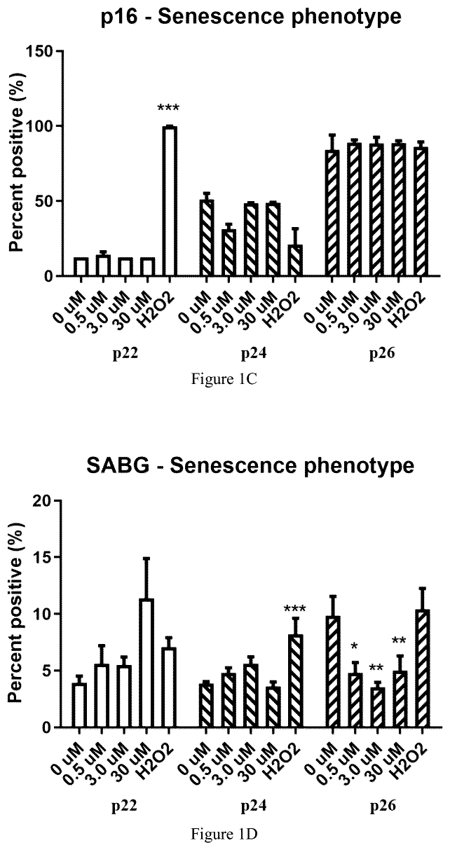

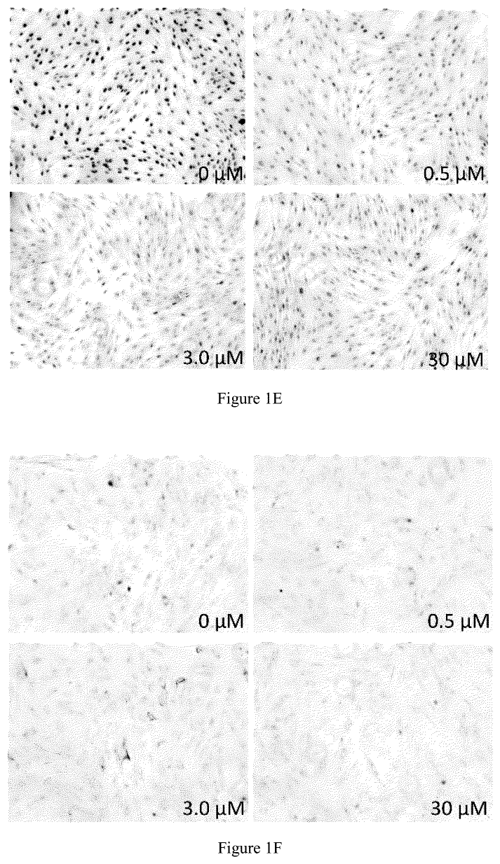

For this assay, HDFn cells chronically treated with lower EGT concentrations (0, 0.5, 3.0 and 30 μM) were analyzed for senescence markers at two passages when the HDFn cells were still proliferative (passage 22 and 24), and at one passage when the HDFn cells were was in replicative senescence stage (passage 26) (FIGS. 1A-1F). Passage 26 was selected for the final passage as the HDFn cells displayed a senescent phenotype for up to a week following passaging, however, replication did continue at a slow rate to passage 31 (data not shown).

The HDFn cells showed a general progression towards replicative senescence between passage 22 to 26; baseline (0 μM EGT treatment) Ki67 positive HDFn cells increased from 20% to 40% of the total cell population between passage 22 to 24 and decreased back to 20% at passage 26 (FIG. 1A). P21 positive HDFn cells increased from 82.5%±2.3 to 94.2%±0.3 (mean±SEM) of the total cell population between passage 22 and 24, and Baseline p21 positive HDFn cells were high across all three passages (FIG. 1B). In contrast, p16 positive HDFn cells showed a passage dependent increase from 12.5% to 51.2%±3.9 to 88.8%±9.9 (mean±SEM) of the total cell population in passage 22, 24, and 26, respectively (FIG. 1C). Baseline senescence-associated β-galactosidase (SAβG) was low across all three passages (3.6%±0.6, 7.1%±0.2, and 9.8%±1.7) (FIG. 1D). This was unexpected since SAβG levels remained low even with hydrogen peroxide treatment.

For all three passages, EGT treatments did not result in statistically significant changes of percentage of Ki67 positive HDFn cells when compared with the untreated control (FIG. 1A). In contrast, EGT reduced the percentage of p21 positive HDFn cells (FIG. 1B) in passage 22 by 16.5% at 0.5 μM (82.5%±2.3 to 66.0%±2.4, p<0.001), 20.1% at 3 μM (p<0.001), and 21.8% at 30 μM (p<0.001) (FIG. 1B). EGT treatments did not result in statistically significant changes of percentage of p21 positive HDFn cells in passage 24 or 26 (FIG. 1B). EGT also did not robustly affect the percentage of p16 positive HDFn cells across all three passages, although there was a 15% reduction in p16-positive cells in passage 24 after 0.5 μM EGT treatment (p<0.001, FIG. 1C). All three concentrations of EGT reduced the percentage of SAβG positive HDFn cells at passage 26: from 9.8%±2.7 to 4.8%±5.7, 3.5%±2.0 and 5.0%±2.9 at 0.5 μM, 3.0 μM, and 30 μM, respectively (p<0.001). While the absolute levels of SAβG-positive cells were low, there is a mean of 50% SAβG reduction.

Upon senescence induction by various inducers, p21 is first recruited to the nucleus to prevent further proliferation. β-galactosidase is a lysosomal enzyme that is upregulated during cessation of cell proliferation. Thus, down-regulation of p21 and/or SAβG could suppress the deleterious effects of senescence. In conclusion, it appears that low chronic doses of L-Ergothioneine can confer senostatic effects in HDFn cells.

### Senolytic Profile Assay

For this assay, HDFn cells acutely treated with higher EGT concentrations were analyzed for cell population counts and senescence markers at passage 31 to determine if EGT can be senolytic. 13 μM of Navitoclax and 600 μM of Fisetin were used as positive controls. Three timepoints were taken for this assay at day 0, 6, and 9 after seeding, plus three days of EGT exposure, to ensure that a senescent HDFn timepoint could be captured.

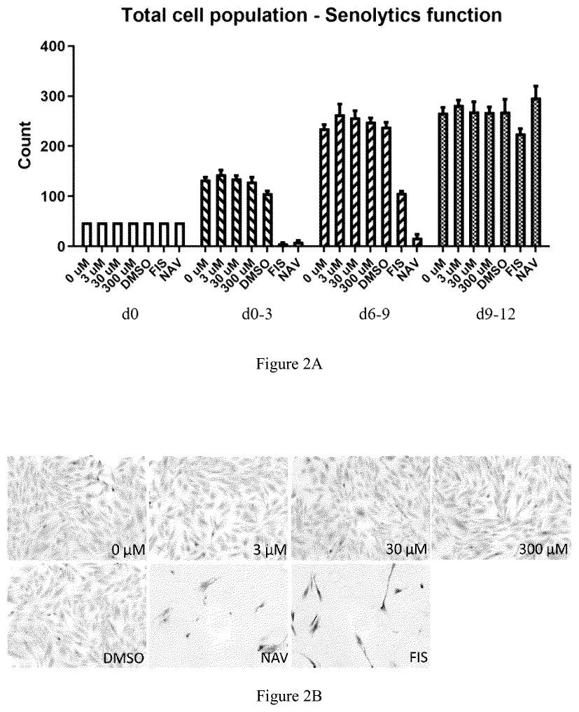

FIG. 2A are bar charts showing cell counts with various treatments. FIG. 2B shows representative images for cell count with various treatments. These data show that cell counts did not change much among 0, 3, 30 and 300 μM of EGT treatments, while cell counts dramatically decreased in positive controls (FIS and NAV in FIG. 2A). The data suggests that EGT does not display senolytic activity when starting treatment of HDFn cells at passage 31.

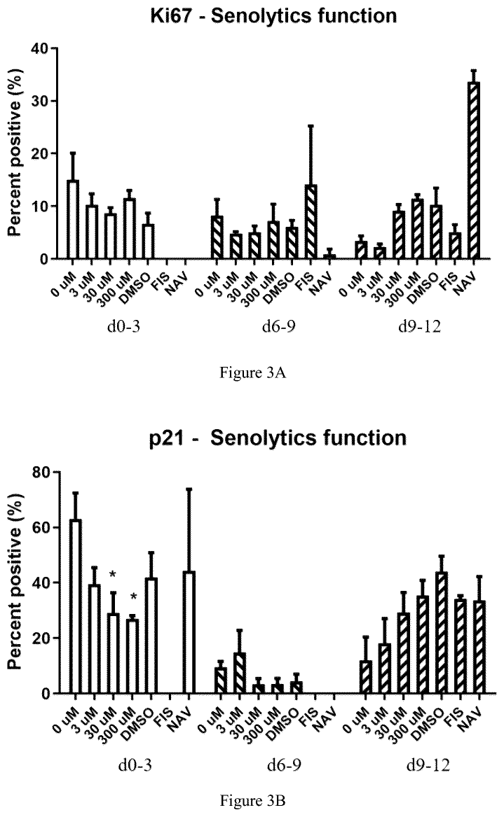

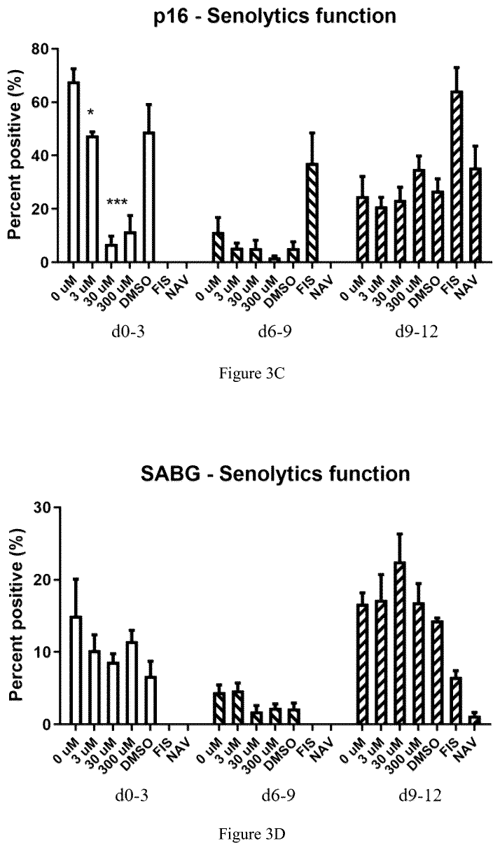

FIGS. 3A-3D show percentage of HDFn cells that are positive for senescence markers at each timepoints. FIG. 3B shows that 30 and 300 μM EGT reduced the percentage of p21 positive HDFn cells from 63.1%±9.3 to 29.1%±7.4 and 27.0%±2.0 respectively at timepoint 1 (day 0 to 3). The percentage of p16 positive HDFn cells were also reduced by 30 and 300 μM EGT from 67.8%±4.7 to 7.0%±2.8 and 11.7%±5.9 for this timepoint (FIG. 3C), while there was no statistically significant effect of EGT on the percentage of SAβBG positive HDFn cells at any timepoint (FIG. 3D). 30 and 300 μM EGT also increased the Ki67-positive population from 3.4%±1.5 to 9.2%±3.8 and 11.4%±2.6 respectively (p<0.001) at the day 9 to 12 timepoint (FIG. 3A). It is possible that this increase in the percentage of Ki67 positive HDFn cells at the later timepoint is an effect of p16 and p21 downregulation at the earlier timepoint. Although a reduction of senescence markers is observed, the number of cells present in the assay remains the same. This means that senolytic activity has not happened since senolytic activity is recognized by the selective clearance of senescence cells.

Taken together, this assay shows that L-Ergothioneine is not senolytic at up to 300 μM.

This study was performed to investigate if the compound Ergothioneine (EGT) showed any senostatic or senolytic activity. Human dermal fibroblast (HDFn) cultures were supplemented with 0.5, 3.0 and 30 μM of L-Ergothioneine and the effect of EGT on replicative senescence was observed. Cells were analyzed for biomarkers of senescence (Ki67, p16, p21, and senescence-associated β-galactosidase) at three selected passages in order to determine senostatic activity. Additionally, once the HDFn cultures reached senescence, they were supplemented with 3, 30 and 300 μM of EGT for three days and analyzed for biomarkers of senescence to determine any potential senolytic effects of EGT.

The data suggests EGT has senostatic activity but does not display senolytic activity. There is currently no reported EGT activity in the caspase pathways through which the best-established senolytics are known to function. Thus, the data shown here is reasonably conform to previous reports.

Current publications on EGT indicate that it is an antioxidant that may function through the p38 MAPK pathway. This may indicate that EGT is inducing senostasis by affecting the NAD cycle (Nacarelli et al, 2019, NAD. Nat Cell Biol, 21 (3), 397-407), in addition to other senostatic functions through the MAPK pathways (Anerillas et al, 2020, Regulation of senescence traits by MAPKs. Geroscience, 42 (2), 397-408). The data shown here provides a basis for further mechanistic studies into the senostatic functions of EGT and to state with certainty that EGT is senostatic.

According to the above data, L-Ergothioneine has putative senostatic function in HDFn, but no senolytic function.

What is claimed is:

1. A method for suppressing senescence in a mammal in need thereof, comprising administration to the mammal of a composition comprising a therapeutically effective amount of ergothioneine or a pharmaceutically acceptable salt, acid, ester, analog or derivative thereof, wherein the suppressing senescence is by down-regulation of p21 and/or SAβG.

2. The method of claim 1, wherein the senescence phenotype is reflected through HDFn.

3. The method of claim 1, wherein the mammal is a human, horse, cattle or other ruminants, pig, or a pet.

4. The method of claim 1, wherein the composition is in a form of food, drink, nutritional composition, or pharmaceutical composition.

5. The method of claim 1, wherein the composition is in a form of solution, liquid suspension, parenteral solution, injectable solution, tablet, pill, granule, powder, film, (micro) capsule, aerosol, tonic, syrup, beverage, or dietary supplement.

6. The method of claim 1, wherein the administration is at least once a day or more times a day.

7. The method of claim 1, wherein the administration is via oral, intravenous injectable, intramuscular injectable, intraperitoneal, intranasal, rectal, or sublingual route.

8. The method of claim 1, wherein the administration of the composition is with a daily dose of ergothioneine in the range of 2-500 mg.

9. The method of claim 8, wherein the daily dose is administered in divided doses or a single dose.

10. The method of claim 1, wherein the administration is for a week or more, or for months or years.
